(19) United States
(12) Patent Application Publication
(10) Pub. No.: US 2010/0036266 A1
(43) Pub. Date: Feb. 11, 2010

(54) DEVICE AND METHOD FOR DETECTING HEART BEATS USING AIRWAY PRESSURE

(75) Inventors: Helge Myklebust, Stavanger (NO); Joar Eilevstjonn, Sandnes (NO); Jon Nysaether, Hafrsfjord (NO)

Correspondence Address: DORSEY & WHITNEY LLP;INTELLECTUAL PROPERTY DEPARTMENT, Columbia Center, 701 Fifth Avenue, Suite 6100, SEATTLE, WA, 98104-7043, US

(73) Assignee: Laerdal Medical AS, Stavanger (NO)

(21) Appl. No.: 12/533,784

(22) Filed: Jul. 31, 2009

(30) Foreign Application Priority Data
Jul. 31, 2008 (GB) 0814066.7

(51) Int. Cl. A61B 5/02

(52) U.S. Cl. 600/500

(57) ABSTRACT

Embodiments are directed to a device and method for detecting heart beats and/or monitoring ventilation and/or respiration of a patient using air pressure. In one embodiment, a monitoring device comprises a gas duct and at least one pressure sensor configured to measure pressure in the duct. The duct may be in fluid communication with a patients airways. Based on the sensed pressure, a heart beat of the patient may be detected. In another embodiment, a flow rate of the air expired from the patient may be calculated based on the pressure and a known flow resistance.

### CROSS-REFERENCE TO RELATED APPLICATION

This application claims priority from United Kingdom Application No. 0814066.7, filed Jul. 31, 2008. This application is incorporated by reference herein.

### TECHNICAL FIELD

This invention is directed to a device and method for obtaining heart beat information and monitoring respiration and/or ventilation in a patient by monitoring the patient's airway pressure.

### BACKGROUND

In many medical situations, such as while resuscitation is being performed on a patient, there may be a need for monitoring a patient's heart beat or pulse beat. Additionally, there is often a need for monitoring a patient's respiration or ventilations being provided to the patient. Such medical situations, for example, might include cardiac arrest, respiratory obstruction, asthma, chronic obstructive pulmonary disease (COPD), heart failure, major trauma, overdose, seizure, sepsis, or during anesthesia.

Traditionally, the heart rate is measured manually through palpation by a person feeling and counting pulse beats on a patient, such as by touching a patient's wrist and counting the pulse beats. More recently, however, a heart rate may be measured by an electrocardiography (ECG) apparatus. An ECG apparatus is typically quite expensive. Furthermore, the method of using an ECG is somewhat complex, requiring that electrodes be connected to various locations on a patient's skin.

Therefore, there is a need for a simple and accurate way to monitor a patient's heart, such as heart beat or pulse beat, during resuscitation and/or ventilation.

### DESCRIPTION OF DRAWINGS

FIGS. 1a-1c are schematic illustrations of a monitoring device according to one embodiment of the invention.

FIG. 2a illustrates a block diagram of a monitoring device used in conjunction with a ventilation system according to one embodiment of the invention.

FIG. 2b is a schematic illustration of a monitoring device integrated with a ventilation system according to one embodiment of the invention.

FIG. 3 shows an example of a pressure signal achieved by a monitoring device according to one embodiment of the invention.

FIG. 4 shows an example of a pressure signal achieved by a monitoring device according to one embodiment of the invention.

FIG. 5 illustrates pressure interactions between heart and lungs.

### DETAILED DESCRIPTION

Embodiments of the present invention are directed toward, for example, a device and method for obtaining hear beat information such as detecting and/or monitoring a patient's heart. More particularly, one or more embodiments are directed to a device and method for detecting heart beats in a patient during resuscitation and/or ventilation by evaluating pressure changes in an airway of the patient. Certain details are set forth below to provide a sufficient understanding of the invention. However, it will be clear to one skilled in the art that the invention may be practiced without these particular details.

Throughout this document, the term respiration will include spontaneous and assisted breathing or respiration, including ventilation. Additionally, the term flow may refer to both volume flow and mass flow.

During resuscitation a patient is often ventilated by providing air to the patient's airways through a respiration device, such as a mask or tube, connected to an oxygen or air source, such as ventilation bag. In many embodiments discussed below, ventilation activity may be monitored by sensors, such as flow or pressure sensors, situated in a ventilation path. In one embodiment, based on a measured pressure change, flow rate may be determined, and thus, a ventilation volume may be determined.

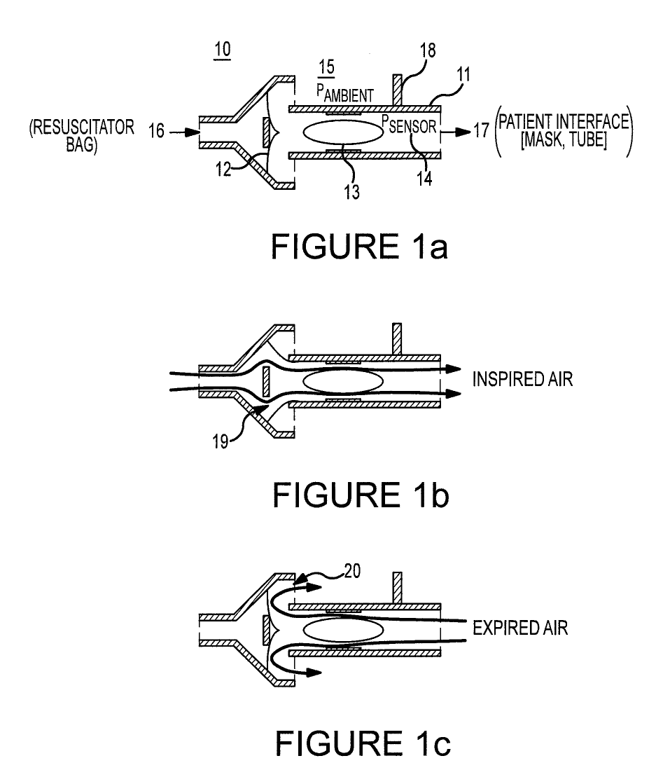

FIGS. 1a-1c are schematic illustrations of a monitoring device 10 according to one embodiment of the invention. The monitoring device 10 may be used in conjunction with a ventilation system. FIG. 1a shows the monitoring device 10 in a rested state. FIG. 1b shows the monitoring device 10 in which air flows from a resuscitator bag 16 through the monitoring device 10 to a patient interface 17 as inspired air. FIG. 1c shows the monitoring device 10 in which air is expired by the patient (not shown) and flows through the monitoring device 10 to an outlet, such as to ambient.

The monitoring device 10 includes a duct 11 operable to pass a gas therethrough. A resuscitator bag or bellow 16 and a face or respiratory mask 17 are connected to the monitoring device 10 on opposite ends of the duct 11. The monitoring device 10 may include at least one pressure sensor 14. The pressure sensor 14 may be configured to measure pressure in the duct 11. The pressure sensor 14 may measure the pressure at a point in which a flow resistance of the passage is known. As will be further explained below, the monitoring device 10 may be operable to provide heart beat information, for example detect a patient's heart beat, and/or measure a volume of air expired from a patient being ventilated, such as by a resuscitator bag 16 and a face mask 17.

The duct 11 may be any duct configured to be arranged in connection with an airway of a patient. In one embodiment, the duct 11 is connected to or integrated in a respiration device, such as a respiratory mask, a tracheal tube, a bellow, an anaesthesia mask, etc. In the embodiment, in which the duct 11 is integrated in a respiration device, the restriction 13 may be a restriction already present in the respiration device, such as a valve, choke, etc.

The monitoring device 10 may include a separate inspiration and expiration flow path. This may be accomplished by a single one-way valve or two separate one-way valves. The purpose of these valves is to allow air from the bag to enter the patient, but not allow expired air from the patient back to the bag. Rather, the expired air is exhausted to ambient through an outlet valve. In the embodiment of FIG. 1, a single one-way valve 12 is arranged as a part of the monitoring device 10. In particular, one-way valve 12, comprising an inlet path 19 (FIG. 1b) and an outlet path 20 (FIG. 1c), is arranged at the end of the duct 11 facing away from the patient. As is illustrated in the FIGS. 1b and 1c, when the one-way valve 12 is positioned to open the inlet path 19, the outlet path 20 is blocked, and when the one-way valve is positioned to open the outlet path 20, the inlet path 19 is blocked. In some embodiments, a one-way valve may be separated from the monitoring device 10 and integrated with the ventilation or resuscitation device, such as, for example, in a resuscitator bag. In these embodiments, the one-way valve(s) may be connected to the monitoring device 10 when in use.

A restriction 13 may be arranged inside the duct 11. The restriction 13 may be operable to restrict gas flow in the duct 11. In some embodiments, the restriction has a known flow resistance. In the embodiment shown in FIGS. 1a-1c, the pressure sensor 14 is positioned on the patient side of the restriction 13. The duct 11 further includes a pressure outlet 18. In one embodiment, the pressure sensor 14 may measure the pressure of the gas on the patient side of the restriction, such as at label Pₛₑₙₛₒᵣ illustrated in FIG. 1a.

In one embodiment, the restriction 13 is shaped to ensure that the flow velocity is substantially equal on both sides of the restriction 13. This may be achieved, for example, by a symmetric shaped restriction. In some embodiments, the restriction 13 may be shaped in such a way that the flow through the restriction is laminar. Additionally, the monitoring device 10 is may be configured such that a low and constant flow rate is provided at the point of pressure measurement. By providing low and constant flow rates, this avoids pressure fluctuations that may compromise the accuracy of flow measurements determined based on pressure measurements made by the pressure sensor 14. In another embodiment, the restriction 13 may be shaped in a way in which the flow through the restriction is turbulent.

The monitoring device 10 may further include a processing unit (not shown) connected to the pressure sensor 14 and arranged for generating heart beat information, for example, detecting heart beat signals in or deriving heart beat signals from pressure signals provided by the pressure sensor. In one embodiment, the processing unit may be configured for calculating flow values from the measured pressure drop. The processing unit may also be configured to calculate respired volume from the flow values or from the measured pressure drop. In one embodiment the processing unit is configured to calculate flow values from a linear relationship between pressure drop and flow.

The monitoring device 10 may also comprise a user interface (not shown) to communicate information to a user regarding measurements made by the pressure sensor 14. For instance, the user interface may be configured to provide information regarding the patient's heart beats using the values of the change in pressure measured by the pressure sensor. Additionally, the user interface may provide information regarding the patient's respiration.

Although FIGS. 1a-1c show the pressure sensor 14 positioned in the duct 11, the physical presence of the pressure sensor 14 may be in an alternative location and connected to the location of pressure measurement by a flexible tube provided through pressure outlet 18. The pressure sensor 14 may be able to detect a change in pressure in the duct 11. For instance, beatings of a patient's heart creates a pressure wave in the patient's torso. This pressure wave leads to a fluctuation in the pressure in the lungs and the airways of the patient. The monitoring device 10, when coupled to a patient via the patient interface 17, receives the pressure waves from the airways of the patient, and the pressure sensor 14 detects the fluctuation in the pressure.

In one embodiment, the pressure drop across the restriction 13 is measured by a single pressure sensor 14 making at least two measurements. In this embodiment, the pressure sensor 14 may be an absolute pressure sensor. In another embodiment, the pressure sensor 14 may be a differential pressure sensor. For instance, a differential pressure sensor may be coupled between a point of pressure measurement and a known reference pressure point, such as ambient. In this way, a flow rate may be calculated directly based on a single pressure measurement. For instance, a pressure drop across the restriction may be measured as a pressure difference between pressure measured by a sensor arranged at one side of the restriction and a known, substantially constant pressure arranged at the other side of the restriction 13, such as ambient pressure. Alternatively, the known, substantially constant pressure may be associated with an overpressure valve. In this case the known pressure will differ from the ambient pressure, by a relatively fixed offset.

In alternative embodiments, the restriction 13 may take many different forms. In some embodiments the restriction may be associated with the outlet path 20, one-way valve 12, or an airway filter (not shown). For instance, if the outlet path 20 has a suitable flow resistance, the outlet path 20 or one-way valve 12 may function as the measurement restriction itself, restriction 13 in FIGS. 1a-1c. In this embodiment, restriction 13 is not needed in the duct 11. Additionally, the restriction 13 and the outlet path 20 may integrated with the resuscitator bag 16.

In another embodiment, the restriction 13, pressure sensor 14, and pressure sensor outlet 18 are integrated in an airway adapter with standardized fittings on both sides. The airway adapter may be for single use or for multiple uses. In use, the airway adapter may be connected between a resuscitator bag on one side and a patient interface, such as a mask or endotracheal tube on the other side. Since the measuring principle is dependent on the flow resistance of an outlet path or one-way outlet valve, this adapter may also comprise a separate outlet valve with a known flow resistance, which blocks the flow to alternative outlet valves with unknown resistance.

A method of using the monitoring device will now be explained. In one embodiment, an elevated pressure may be provided in the lungs of the patient for a brief period. This may, for example, be done by a ventilation bag, external air, or an oxygen source, which supplies air or oxygen to the lungs through the airway of the patient. In this way, the lungs expand towards the heart, providing a tighter contact between the lungs and heart, and thus a better transmission of the pressure from the heart beat to the airway.

As is discussed above, the flow restriction 13 is placed between the pressure sensor outlet 18 and the outlet path 20. Thus, during expiration, air flows from the patient, past the pressure measurement location A through the restriction 13 and out of the outlet path 20. The pressure drop from the pressure measurement location A to ambient is thus determined by the flow resistance through the restriction 13 and the outlet path 20 in series. If the total resistance of this series flow restriction, such as the resistance between the pressure sensor and ambient, is known, the overpressure (over ambient) measured by the pressure sensor can be used to determine the flow through the restriction. By integrating the measured flow during expiration, the expired volume may be calculated. The expired volume is indicative of how much air has actually gone into the lungs. In order to ensure that the total restriction resistance is known, there may be provided an outlet valve with known characteristics, such as flow resistance, connected to the duct 11.

As will be clear to those skilled in the art, the principle described above is fundamentally different from the venturi principle. Although both principles make use of a restriction, the purpose of the restriction in the venturi principle is to accelerate the air flow and detect the reduction in pressure that follows from increased air speed through Bernoulli's theorem. In the current device, however, the purpose of the restriction is similar to that of a resistor in an electrical circuit. In an analogy with a voltage drop across a resistor, which is proportional to the electrical current, the pressure drop across a restriction will be proportional to the flow through the restriction (provided that the air speed is similar at both ends of the restriction). This linear relationship between pressure drop and flow is an advantage of the monitoring device over the venturi principle, where there is a quadratic relationship.

In addition to providing a measurement of flow, and thus volume, measurements from the pressure sensor may be used to detect other important events and situations associated with ventilation and/or respiration. For instance, if the measured pressure attains elevated values during inspiration, while the measured expiration volume is low, this may be an indication that the airway is occluded. Ventilation parameters such as ventilation rate, inspiration time, and expiration time may also be derived from the pressure reading. Additionally, detecting and monitoring heart beats are also possible based on the measurements from the pressure sensor.

By observing the maximum airway pressure and the corresponding expired volume, the compliance of the lung can be estimated. By observing the flow at the beginning of the expiration phase, the airway resistance may also be calculated. Based on these two parameters, it may be possible to estimate the inspiration flow from the measured pressure. This estimated flow can for instance be used to generate a volume or flow waveform of the entire ventilation, and not only of the expiration phase. Also, if the measured expired volume is significantly lower than the calculated inspired volume, mask leakage may be indicated.

Yet another use of the reading from the pressure sensor is to evaluate compression parameters from chest compressions performed during CPR (Cardiopulmonary Resuscitation). The compressions performed on the chest of a patient will be present as pressure peaks in the airway pressure signal. From this, it can be calculated parameters such as compression rate, time period without compressions, relative force used on compression, etc. The pressure peaks will have similar characteristics to the pressure signal representing the heart beats, and the calculations may comprise differentiating these two signals, or the user may be able to differentiate the signals manually.

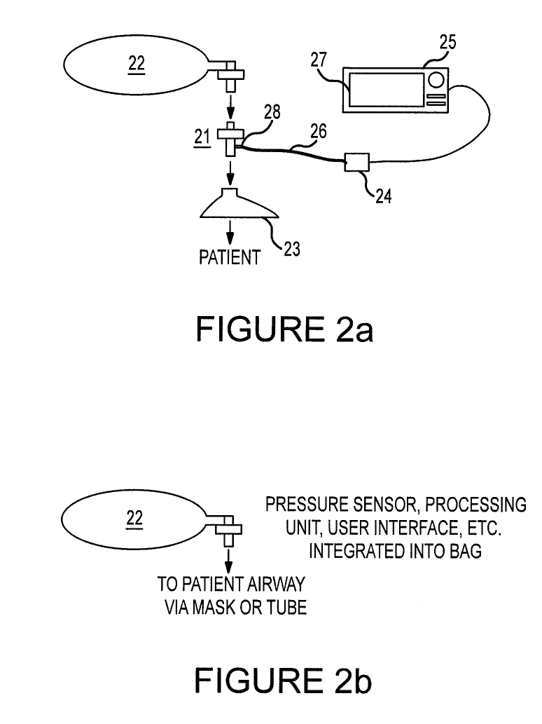

FIG. 2a illustrates a block diagram of a system comprising a monitoring device and a ventilation device according to one embodiment of the invention. The system includes a processing unit 25 and a user interface 27. The processing unit 25 comprises required electronics, such as a processor. The user interface 27 may, for example, comprise an output device such as display, light emitting diodes, loudspeaker, etc. In this embodiment, a restriction (not shown), pressure outlet 28 and one-way valve (not shown) are integrated in or a part of airway adapter 21. The airway adapter 21 may be for single-use or may be made for multiple-uses. The airway adapter 21 may be connected at one end to a patient interface, such as a face mask 23, and at another end, to an air or oxygen source, such as a resuscitator bag 22. In some embodiments, airway filters (not shown), such as moisture filters, may be placed between the airway adapter and face mask 23. A pressure sensor 24 is arranged connected to the pressure outlet 28 through tube 26. In one embodiment, the pressure sensor 24 measures the pressure at a location or point on the patient side of the restriction as described in reference to FIGS. 1a-1c. The pressure sensor 24 is connected to the processing unit 25.

In this embodiment, the processing unit 25 comprises the user interface 27. However, as will be clear to a person having ordinary skill in the art, the user interface 27 may be separate from the processing unit. Additionally, the processing unit 25 may be part of a patient monitor and/or defibrillator system (or e.g. AED, compression machine, CPR assist/guidance device, machine ventilator) or it may be a stand-alone unit. The processing unit 25 is configured to receive a signal from the pressure sensor 24 indicative of a pressure or change in pressure measured by the pressure sensor. The processing unit may be configured to provide feedback and/or guidance on ventilation performance to the rescuer and to store data for later use. Such feedback may include numeric values, text, graphs or graphics on a display (visual feedback) or sound or voice prompts through a loudspeaker (audible feedback).

The pressure sensor 24 may be located in the processing unit 25, the airway adapter 21 or as a separate unit (as is illustrated in FIG. 2a). Depending on the location of the pressure sensor, the pressure sensor 24 may be coupled to the processing unit 25 by a variety of ways. For instance, when the pressure sensor is part of the airway adaptor or as a separate, the pressure sensor may be electrically coupled to the pressure sensor via a wire or wirelessly. The pressure sensor 24 may be mechanical and coupled to a transducer that is connected to the processing unit 25.

FIG. 2b illustrates a block diagram of a system including a monitoring device used in conjunction with a ventilation system according to another embodiment of the invention. In this embodiment, the pressure sensor, processing unit, and user interface may be all integrated in a resuscitator bag 22. The resuscitator bag may further include a restriction. These component are similar in function to the components discussed in reference to FIG. 1a and will therefore not be discussed in detail in the interest of brevity.

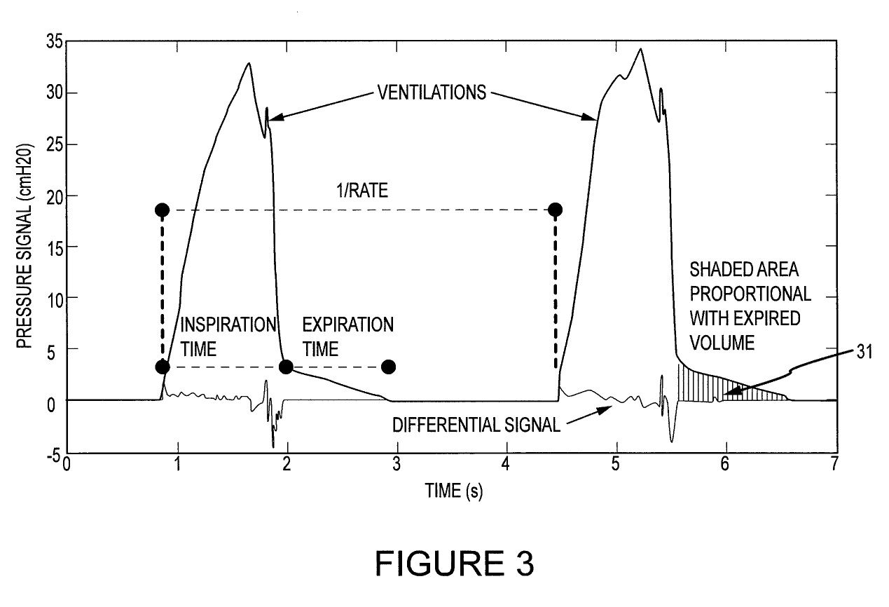

FIG. 3 shows a graph ploting pressure signals provided by a pressure sensor over time according to one embodiment of the invention. The pressure sensor that provides the pressure signals in the graph may be a pressure sensor as is described in reference to FIG. 1 or 2. The pressure sensor 24 may provide a pressure signal indicative of a patient's heart beat and thus, the patient's heart rate may be calculated or derived. In the case where only the heart beat detection is of interest, a simple pressure measurement is sufficient and the restriction may be omitted.

The pressure signal may also detect ventilations and ventilation parameters may be calculated. For instance, in one embodiment parameters associated with ventilation may be determined based on the pressure signal as outlined in the following:
1. Ventilation parameters may be determined, such as through simple time-domain thresholding of the pressure signal.
2. The ventilation rate may be the inverse of the time between two ventilations.
3. The expired volume of a ventilation is proportional to the area 31 shaded in FIG. 3 (the area under the ventilation pressure signal after the initial rapid decrease in pressure until pressure reaches a zero level). This area may be found by integration, such as in a digitized signal summing up the samples from an integration start time to an integration stop time. To find the integration start time, the differential signal can be used (i.e. the first derivative of the pressure signal). When the differential signal goes below a negative threshold, the integration start time can be set when the differential signal returns to a near zero threshold. Alternatively the integration start time could be when the pressure goes below a certain percentage of peak ventilation pressure. Integration can be stopped when the pressure signal reaches a near zero/baseline value. A timeout can be used if the pressure signal for some reason never reaches this near zero/baseline value, either to stop or cancel integration. The area calculated through integration is approximately linearly proportional with the volume of the expired air. The proportionality coefficient can be found through calibration of the system.
4. The inspiration time (inflation/insufflation time) may be determined by taking the time from start of ventilation (e.g. the detection threshold) to the integration start time.
5. The expiration time may be determined by taking the time from integration start to integration stop.

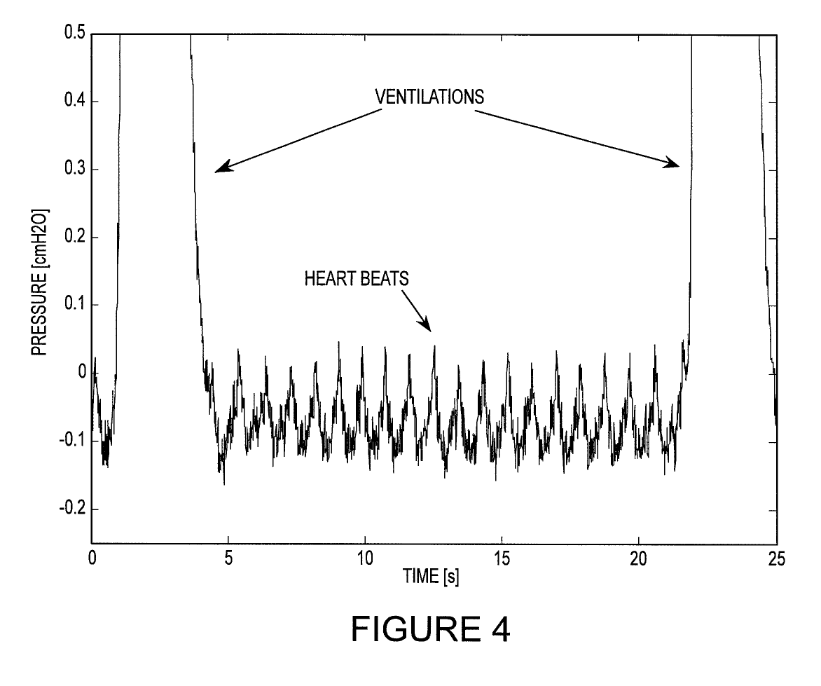

FIG. 4 shows a graph plotting pressure signals provided by a pressure sensor over time according to another embodiment of the invention. The pressure sensor may be a pressure sensor as is described in reference to FIG. 1 or 2. In this view the heartbeats are visible as the small peaks between the larger peaks, which represents ventilations. A heart beat may be detected or calculated through simple time-domain thresholding of the pressure signal in a similar way as ventilations. The heart rate/pulse rate will then be the inverse of the time between two heart beats. Other methods can also be used, such as autocorrelation or frequency domain based methods, such as spectrum.

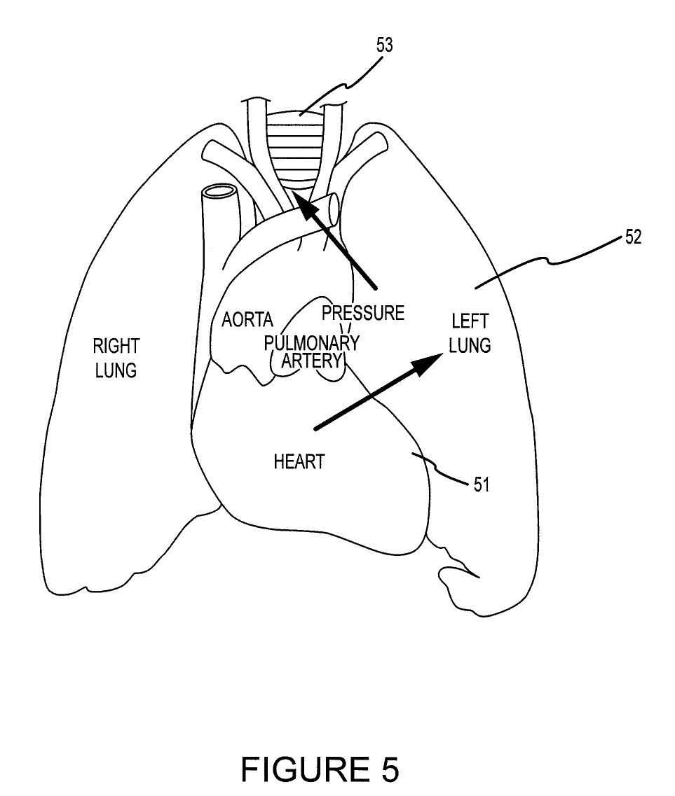

FIG. 5 is schematic figure illustrating the pressure interaction between a heart 51 and lungs 52 of a patient. When the pressure in the lungs 52 is elevated, the lungs 52 expand and come closer into contact with the heart 51. When the heart 51 beats, the movement and/or force of the beats will be transferred to the lungs, thus making a pressure fluctuation or small pressure wave in the lungs, which in turn is transferred to the airway or trachea 53 of the patient. The heart beats can thus be detected by the pressure sensor 14 (FIG. 1a), which is connected to the airways.

In one embodiment, the pressure sensor may be configured to measure spontaneous respiration. In this embodiment, the sensor and processing unit in the embodiments of FIGS. 1 and 2 may be used in conjunction with a ventilation monitor. However, the calculations might be altered since the pressures associated with respiration are somewhat different from those for ventilation. A patient's own respirations will have smaller peak pressures than a ventilation and will also be bipolar. For instance, during the inspiration phase the pressure will be negative and during the expiration phase the pressure will be positive. The respiration detection algorithm can then utilize and combine both a negative and positive pressure threshold.

In alternative embodiment, a pressure sensor may be integrated in a simple mask, such as used in supplying oxygen to a patient with respiratory problems, but not respiratory arrest. In this embodiment, pressure may be measured inside the mask. If the flow resistance from the mask to ambient is known, this could work as the restriction and thus enable expiration volume calculation. However, in any case the pressure signal provided will be sufficient to monitor other parameters such as respiration rate, and most importantly, to monitor whether the patient breathes or not.

Embodiments may also be combined with additional sensors to provide better assessment of the status of the patient and to provide feedback of rescue efforts. Such additional sensors may include, for example, SPO₂, ETCO₂, ECG, impedance, and a compression sensor.

Although the present invention has been described with reference to the disclosed embodiments, persons skilled in the art will recognize that changes may be made in form and detail without departing from the spirit and scope of the claims. Such modifications are well within the skill of those ordinarily skilled in the art. Accordingly, the invention is not limited except as by the appended claims.

What is claimed is:

1. A device for obtaining heart beat information of a patient, comprising:
a duct configured to allow a gas to pass therethrough;
at least one pressure sensor connected to the duct and configured to measure the pressure in the duct; and
a processing unit connected to the pressure sensor and operable to generate heart beat information from the pressure signal.

2. The device according to claim 1, wherein the duct is connected to a respiration device.

3. The device according to claim 1, wherein the duct is integrated in a respiration device.

4. The device according to claim 1, further comprising a restriction arranged in the duct, and wherein the pressure sensor is configured to measure a pressure drop across the restriction as a pressure difference between a location in the duct at a first side of the restriction and a known, substantially constant pressure, arranged at a second side of the restriction.

5. The device according to claim 1 further comprising a one-way valve with known flow resistance connected to the duct.

6. The device according to claim 2 wherein the respiration device comprises a respiratory mask or a tracheal tube.

7. The device according to claim 3, wherein the respiration device comprises a bellow.

8. The device according to claim 4, wherein the pressure drop is measured by a single pressure sensor.

9. The device according claim 1, further comprising a restriction arranged in the duct and wherein the pressure sensor is arranged to measure pressure at a location at a first side of the restriction.

10. The device according to claim 1, further comprising a restriction having a first and second side arranged in the duct and wherein the restriction is shaped to ensure that a flow velocity is substantially equal on the first and second sides of the restriction.

11. The device according to claim 1, wherein the pressure sensor is an absolute pressure sensor.

12. The device according to claim 1, wherein the pressure sensor is a differential pressure sensor.

13. The device according to claim 4, wherein the known, substantially constant pressure, is ambient pressure.

14. The device according to claim 1, wherein the processing unit is adapted to calculate flow values from a linear relationship between pressure drop and flow.

15. The device according to claim 1, wherein the processing unit is adapted to calculate respired volume.

16. A method for obtaining heart beat information of a patient, comprising:
measuring pressure in a gas duct connected to a patient's airways; and
deriving patient heart beat information based on the measured pressure.

17. The method according to claim 16, further comprising providing an elevated pressure in the lungs of the patient.

18. The method according to claim 16, further comprising connecting the duct to a respiration device.

19. The method according to claim 16, wherein the step of measuring pressure in a gas duct comprises measuring a pressure drop across a restriction as the difference between a pressure measured at a location in the duct at a first side of the restriction and a known, substantially constant pressure, arranged at a second side of the restriction.

20. The method according to claim 19, wherein measuring the pressure drop is by a single pressure sensor.

21. The method according to claim 19, wherein the pressure sensor is arranged at the first side of the restriction.

22. The method according to claim 19, wherein the flow velocity is substantially equal on both sides of the restriction.

23. The method according to claim 17, further comprising calculating flow values from a linear relationship between pressure drop and flow.
